(19) United States
(12) Patent Application Publication
(10) Pub. No.: US 2008/0190220 A1
(43) Pub. Date: Aug. 14, 2008

(54) Novel Microfluidic Sample Holder

(76) Inventors: Oktavia Backes, Remagen (DE); Perdita Backes, Remagen (DE)

Correspondence Address: BUCHANAN, INGERSOLL & ROONEY PC, POST OFFICE BOX 1404, ALEXANDRIA, VA, 22313-1404, US

(21) Appl. No.: 11/793,808

(22) PCT Filed: Dec. 23, 2005
(86) PCT No.: PCT/EP2005/014000

§ 371 Date: Feb. 18, 2008

(30) Foreign Application Priority Data
Dec. 23, 2004 (DE) 10 2004 063 438.6

(51) Int. Cl. G01N 1/00; B01J 19/00; C12M 1/40

(52) U.S. Cl. 73/864.81; 422/129; 422/61; 435/289.1

(57) ABSTRACT

A novel microfluidic sample holder has at least one sample receiving compartment for a sample fluid, at least one distributor channel which is linked with at least one sample receiving compartment, at least one distributor channel extending from every sample receiving compartment, at least one reaction chamber to which optionally one inlet channel branched off from the at least one distributor channel leads, and at least one vent opening for every reaction chamber. The sample holders are mainly used in microbiological diagnostics, immunology, PCR, clinical chemistry, microanalytics and/or the inspection of active substances. Methods for analyzing a sample substance using the sample holder and to kits including the sample holder as also included.

### FIELD OF THE INVENTION

The invention relates to a novel sample holder having at least one sample receiving chamber for a sample fluid, at least one distributor channel that is connected to the at least one sample receiving chamber, at least one distributor channel extending from each sample receiving chamber, at least one reaction chamber into which, if appropriate, an inlet channel branching off from the at least one distributor channel opens, and at least one vent opening for each reaction chamber. Such sample holders serve chiefly for use in microbiological diagnostics, immunology, PCR, clinical chemistry, microanalytics and/or the testing of active substances. The invention further relates to methods for analyzing a sample substance in which the sample holder is used, and to kits that include the sample holder.

### PRIOR ART

The enormous advances in the development of biochips also opens up new dimensions in medical diagnostics. In view of the growing problems of financing public health, particular importance attaches here chiefly to the aspect of possible savings in cost. Scientific and technological development has brought forth many approaches in years past as to how diagnostic questions can be modified with the aid of multiparameter tests. The greatest success here has been the development in the field of so called biochips, in particular in the area of DNA chips. Other test formats have been developed in parallel therewith, for example bead technologies and microfluidic systems.

Microfluidics is generally understood as the handling and management of very small fluid quantities (for example microliters, nanoliters or even picoliters). Various methods can be used for the targeted movement of fluids:
electrokinetics
pressure
capillarity.

These can be applied individually, or else in combination. The electrokinetic flow is achieved in this case by applying electric voltage to the channels. The phenomena that occur, known as electroosmosis and electrophoresis, lead to the movement of charged molecules. By contrast therewith, it is also possible for uncharged molecules and, for example, cells to be moved by applying pressures (for example with micropumps). Passive movement is increasingly being used alongside these active methods. In this case, capillary force can be employed to move the fluids in a targeted fashion. An important advantage of this technique is that it manages without further drive mechanisms, and therefore enables a drastic simplification of the overall system.

Seen in global terms, most approaches to solutions concentrate of “active elements” for transporting fluids. The structures required in this case are overwhelmingly produced by laser ablation or by hot stamping or injection molding. This restricts the possibilities of structuring in many instances. First approaches for solving passive transport of fluids already exist in Germany. In these instances, the molded part has so far been produced by microinjection molding, and the energy required for transporting fluids has so far been provided by hydrophilization of the surface by means of plasma treatment. A disadvantage of this technique is a tendency of thin hydrophilized layers to anisotrophy of the surfaces (aging through hydrophobic recovery), and their relatively high sensitivity to chemicals and solvents. A method based on photolithography can provide an alternative. Here, the structures are produced with the aid of optical masks by optical polymerization of acrylates. Copolymers with targeted surface properties can be produced by adding suitable crosslinkable organic substances. Moreover, this method omits the production of three-dimensional structures that cannot be implemented with other methods, or can be implemented only at an unacceptable cost.

Such a sample holder that uses only capillary forces to transport sample fluids is known, for example, from WO 99/46045. What is involved here are plastic chips that are produced using the microinjection molding method and are subsequently modified (hydrophilized) by plasma treatment or grafting of surfaces. These methods are expensive and have a range of disadvantages:
1. The surface modification cannot be maintained for a sufficient length of time because of hydrophobic recovery and, in addition, it is not possible to control the homogeneity in a three dimensional direction.
2. Particularly in the case of the assembly of tests, an excessively high hydrophilization effects an undesired back capillarization of substances into the inlet and the vent capillaries, with the risk of blocking the capillaries. The sample holder thus becomes unusable.
3. Capillaries can easily be formed between the walls of the depression and the cover (in particular, given inadequate sealing or use of hydrophilic adhesives) at the inlet points into the test depressions, the consequence being that the depression is not filled, or is filled incompletely, since the fluid flows directly into the vent structure and fills this up such that neighboring depressions can no longer be filled because of a lack of venting. Moreover, in such an instance the fluid can capillarize over the outer edge of the sample holder into further vent structures belonging to other tests.
4. A high, free surface energy of the sample holder that is intended to ensure the transport of fluids into the test depression is, however, very sensitive to surfactants in fluids, since the abovedescribed faults occur in an intensified fashion here. Consequently, many possible applications are excluded, since, in particular, nonionic surfactants are indispensable in many diagnostic assays (immunoassay, DNA assays, clinical chemistry).
5. The above described sample holder can be used only for single step assays.

Microfluidic chips and/or sample holders offer the possibility of substantially scaling down diagnostic methods and at the same time raising the sample throughput. On the basis of the reduction, it is possible to attain faster reactions, high sensitivities and better control over the sequences by comparison with conventional methods. The development of a reliably functioning microfluidic chip or sample holder is therefore a decisive milestone on the way to an innovative, miniaturized diagnostic system.

Microfluidic chips or sample holders include three-dimensional elements of very different dimensioning. Thus, for example, as a transition from capillary and the “reaction cavity” it is necessary for the laminar fluid flow to be directed toward the bottom of the vessel in order to fill the latter completely. Because of further capillarities, which are formed, inter alia, by the cover and the sidewalls of the reaction vessel, there is, the possibility of other flow directions. Consequently, chaotic flow that cannot be controlled is expected at this transition. A microfluidic structure that reliably—even under the most adverse conditions—reliably ensures a complete filling of reaction cavities is therefore mandatory but, so far, not present.

### OBJECT

The invention addresses the object of providing a novel sample holder, methods for analyzing a sample substance with the use of the novel sample holder, and kits containing the novel sample holder that assist in overcoming the disadvantages present in the prior art, in particular in improving the filling dynamics, reducing the poor sensitivity, providing by simple and cost effective devices the possibility of carrying out one-step or, for example, multistep assays, and which are specific and sensitive enough to ensure a fast, quantitative identification of the sample substance.

### Achievement

This object is achieved by the invention having the features of the independent claim. Advantageous developments of the invention are characterized in the subclaims. The wording of all the claims is hereby incorporated in the description by reference.

It was possible to provide an improved microfluidic sample holder through a range of measures that relate both to the geometry of the structures, the arrangement of certain structural elements, the use of gradients of the free surface energy in a vertical direction, and also to the nonionic surfactants, suitable for the application, in the sample fluids and during test assembly. The following crucial points, in particular, were put in this case:
novel design of the distribution channels and ventilation channels
research into the influence of specific dimensions (height of the structures)
novel design of the capillary stop structures
research into the influence of the surface energy of the fluid on the flow behavior
statistical analysis of the filling time.

Individual method steps are described in more detail below. The steps need not necessarily be carried out in the specified sequence, and the method to be outlined can also have further, unnamed steps.

Provision is made of a sample holder having at least one sample receiving chamber for a sample fluid, at least one distributor channel that is connected to the at least one sample receiving chamber, at least one distributor channel extending from each sample receiving chamber, at least one reaction chamber into which, if appropriate, an inlet channel branching off from the at least one distributor channel opens, and at least one vent opening for each reaction chamber. Between the sample receiving chamber, distributor channel, reaction chamber, inlet channel, present if appropriate, and/or the vent channel this sample holder has at least one further additional structure that is at least partially of hydrophobic design. This structure, which is intended, on the one hand, to enable the escape of the air displaced by the inflowing fluid (sample receiving chamber→distributor channel→reaction chamber) and, on the other hand, to cancel or acutely retard the capillary action (capillary stop), can, if appropriate, also be of completely hydrophobic design. These structures can preferably be of relatively small size, for example each further structure can have a cross section of approximately 10 μm to approximately 300 μm, preferably approximately 50 μm to approximately 200 μm, in particular approximately 100 μm to approximately 150 μm. It is important to point out in this context that these structures are in no case to be selected to be so small that they are blocked when a cover element is put on, as is described later. It is important to seal the sample holder (if appropriate, after introducing reagents) for the purpose of a sufficient capillary force for the passive transport of sample fluids in microfluidic sample holders, it being intended for the sample holder not to be blocked by a means possibly used, for example adhesive, when the cover element is put on.

In a preferred embodiment, the additional structure is a substantially semicircular depression that is preferably arranged diagonally opposite the distributor channel. At least one further capillary preferably extends from this semicircular depression, the further capillary being designed in a fashion sharply angled away, preferably at an angle ≧90°, and/or in a zigzag fashion. This further capillary, which can be located on the wall of the distributor channel, retards the fluid flow or brings it to a stop because of the capillary structure. Proceeding from this further capillary, in a further preferred embodiment there extends at least one further element which is substantially sharp edged and has a changing structural depth that can strengthen the aforementioned effects.

It is advantageous when at least one further capillary extends away from the element which is substantially sharp edged and has a changing structural depth, the further capillary opening directly or via a neighboring structure into a terminal depression having a valve function. This neighboring structure can, for example, be a common main vent channel that opens in at least one vent opening. If this further capillary has, for example, been sealed with the aid of a foil no pressure compensation takes place, and so (all) capillary forces potentially cancel one another out. If the seal is opened (for example by puncturing the foil or using a focal laser), the structure fulfils its intended purpose, that is to say filling with the aid of capillary forces begins or continues. The vent structure can also proceed from a distributor channel and/or an inlet channel that interconnects the various structures, for example the sample receiving chamber, the distributor channel, the reaction chamber, the inlet channels, additional structures etc., in the case of which the vent structure/vent opening is initially closed. In this case, the open vent structure of the first test depression (for example first reaction chamber) ends at the side thereof. If a sample substance is then applied thereto, the first depression is filled such that the first step of a reaction can run. Thereafter, the vent system of the second test depression (for example second reaction chamber), which preferably has a lesser volume, is opened and filled from the first depression with the sample substance, now altered. A second step of a reaction can run.

In a further preferred embodiment, in its upper region the inlet channel lies in a plane with the vent opening. The inlet channel is preferably of substantially hydrophobic design in this region. The lower region of the inlet channel, that is to say the region that lies beneath the plane of the vent opening, is preferably substantially of hydrophilic design. As an alternative thereto, only the bottom of the inlet channel can be fabricated from a more hydrophilic material (compared to the material used in the upper region). In WO 99/46045, the sample distribution is performed via a distributor channel that proceeds from a sample application point and branches off from the inlet channels to the test depressions (for example reaction chambers). Such systems for sample distribution are also known from other applications, but for the reasons mentioned above these systems are unsuitable for ensuring an adequate filling dynamics. It is therefore preferably possible for the inlet channels and/or the distributor channels also to proceed individually from the sample receiving chamber. Furthermore, the distributor channel, which is connected to the sample receiving chamber, can preferably be of meandering design and be connected to the sample receiving chamber directly (that is to say without interposition of an inlet channel branching off from it). Of course, the function of the distributor channel can be taken over or supplemented by an inlet channel that may be present such that meandering configurations on the distributor channel and/or the inlet channel are likewise covered by the invention. Furthermore, a number of vent openings, distributor channels, if appropriate inlet channels, reaction chambers and/or additional structures can preferably be arranged around the sample receiving chamber of parallel thereto. Such configurations comprise, for example, “jellyfish forms”, the function of the “jellyfish head” being taken over by the sample receiving chamber, and the “jellyfish tentacles” being taken over by the distributor and/or inlet channels. According to the invention, it is likewise provided that the sample receiving chamber is formed centrally as a circle or an ellipse or an elongated structure (so called “arthropod structure”), and the distributor and/or inlet channels (and/or the additional structures) depart therefrom. Arrangements enabling two-step or multistep assays can be arranged correspondingly.

In an advantageous development of the invention, the reaction chamber has a vertical extent of approximately 500 μm to approximately 3 mm, preferably approximately 1 mm to approximately 2.5 mm, in particular approximately 1.5 mm to approximately 2 mm. The edge length of the reaction chamber has an average of approximately 300 μm to approximately 1 mm, preferably approximately 500 μm to approximately 750 μm, in particular 500 μm to approximately 600 μm. The cross section of the reaction chamber is preferably of round, pear shaped, hexahedral, octahedral and/or rectangular design in its cross section. The reaction chamber preferably has a vertically running and substantially rounded inlet capillary in the bottom region, which preferably has a radius of approximately 5 μm to approximately 50 μm, in particular approximately 10 μm to approximately 20 μm. An acute angled inlet capillary seems to be less well suited, since its sharp edges act like a capillary stop and at least retard the fluid flow (or put an end to it completely). It is advantageous when the reaction chamber has an indentation that is preferably arranged diagonally opposite the inlet capillary and leads to at least one vent opening.

In a particularly preferred embodiment, it is provided that in its upper region lying in a plane with the hydrophobic part of the inlet channel the reaction chamber is of substantially hydrophobic design, whereas it is lower region, lying beneath the hydrophobic region of the inlet channel, it is of substantially hydrophilic design. Of course, it is possible thereby for the function of the inlet channel to be taken over or supplemented anew by a distributor channel, as is generally to be pointed out that in all the embodiments of the invention the distributor and inlet channel can supplement one another, that is to say the sample holder has both at least one distributor channel and at least one inlet channel, or the function of the distributor or inlet channel is taken over by at least one channel, that is to say the sample holder has either only at least one distributor channel or only at least one inlet channel. Furthermore, the invention covers any desired combinations between reaction chamber, inlet channel and/or distributor channel. As described, the invention provides that the lower region of the reaction chamber is of hydrophilic design, specifically preferably in such a way that the hydrophilization increases in layerwise fashion. However, it can be necessary under specific conditions for the lower part of the reaction chamber to be at least partially of a (likewise) hydrophobic design. This is advantageous, for example, whenever solutions for drying are applied that contain detergents for improving the solubility of sample substances, something which can lead to strong reverse capillarizations in the case of hydrophilic surfaces. In order to avoid these effects, the reaction chamber can be of generally hydrophobic design in one development of the invention. Owing to the drying of the solution, the detergents then form a hydrophilic film on the hydrophobic surface. The reaction chamber preferably has at least one rounded corner. Again, all the corners of the reaction chamber (with the exception of the corner having the inlet capillary) can be rounded. The capillary force is strongly inhibited by this design of the corners of the reaction chamber, something which once again drastically improves the filling dynamics (radius≧100 μm). It is, furthermore, provided according to the invention that the reaction chamber has sidewalls of substantially smooth and/or corrugated design. It is possible in this case for the sidewalls of corrugated design (radius preferably approximately 30 μm to 50 μm) to act as vertical capillaries while there is a simultaneous enlargement of the surface owing to the corrugated structure. Owing to this arrangement, it is possible when introducing sample substances into solution for the latter to be distributed quickly and uniformly over a relatively large surface in order thus to accelerate the drying process in conjunction with “relief” of the inlet capillaries. The resolubility in the event of addition of the sample substance is also improved. The corrugated structure of the sidewalls can extend over various regions of the walls. Thus, for example, the corrugated structure can extend in the vicinity of the inlet capillaries from the bottom up to the cover, while it is entirely lacking in the vicinity of the vent structure. It has proved that in the case of such a distribution of the corrugated structure the incoming fluid in the region of the inlet capillaries and of the continuous corrugated structure wets the cover element, and the retardation effect in the remaining part is so strong that the air has enough time to escape. Jagged structures appear to be disadvantageous since they cannot be guided down to the bottom because they would disturb the wetting of the bottom.

In a further advantageous development of the invention, it is provided that the sample holder is covered in a fluid-tight fashion by a cover element. As already mentioned, in addition to the suitable geometry of the capillaries it is also important for the sample holder to be sufficiently well sealed (if appropriate after introduction of the sample substances and/or reagents), in order to achieve an adequate capillary force for passively transporting sample fluids in microfluidic sample holders. The cover element is preferably a film that is provided on one side with an adhesive layer of suitable thickness. The film and/or adhesive is preferably a heat activatable and/or pressure sensitive film or adhesive. So far, it has been assumed that strongly hydrophobic adhesives (for example silicone, rubber or silicone rubber adhesives) disturb the fluidics in the capillaries, since said adhesives are still more hydrophobic than plastics not subjected to surface treatment that are usually employed in diagnostics or medical technology (polystyrene, polypropylene, polycarbonate, PMMA). It is a merit of the present invention to demonstrate that precisely these adhesives are particularly suitable for sealing with a cover element. Consequently, in a particularly preferred embodiment a fluoropolymer film is used as film, since its uncoated surface averted from the sample holder is very hydrophobic, has good sliding properties and, something which is advantageous in the case of optical measuring methods, has very strong antisoiling properties. The film is preferably applied under pressure, preferably at approximately 2 to 5 bars, by means of rolls such that the sample holder has a substantially gapless covering. The adhesives are preferably cohesion adhesives. Cohesion adhesives have the property of avoiding “free spaces” under pressure. This is used, for example, in everyday life for the purpose of pointing gaps. In the case of sealing (provided the pressure is not too great, and the adhesive layer is not too thick) this effect can be used advantageously to prevent undesired capillary forces between sidewalls and covering. It has emerged that during sealing of the sample holder “microbeads” form at this site and, together with the hydrophobic properties, prevent this effect (capillarization between sidewalls and cover). Moreover, the adhesive layer is wetted only with a delay, and so during the filling of the test depressions (for example sample receiving chamber, reaction chamber etc.) air has enough time to escape on the vent structure opposite the filling side before said structure is reached by the sample fluid whereupon air bubbles would then be enclosed in the test depression (for example sample receiving chamber, reaction chamber etc.). Hydrophobic adhesives have proved to be particularly suitable adhesives. Such adhesives are, for example, the already mentioned silicone, rubber, silicone rubber and/or fluoropolymer adhesives.

In a further preferred embodiment of the invention, the sample holders according to the invention are used in microbiological diagnostics, immunology, PCR (polymerase chain reaction), clinical chemistry, microanalytics and/or the testing of active substances.

Furthermore, the invention provides a method for analyzing at least one sample substance in the case of which a sample medium has at least one surfactant added to it and is applied to a sample holder according to the invention. This surfactant is preferably a non-ionic surfactant. This nonionic surfactant is preferably a substance whose HLB (hydrophilic-lipophilic balance) number is between approximately 9 to approximately 13. Such surfactants are preferably propylene oxide/ethylene oxide triblock polymers, alkyl polyglycosides, nonyl phenylethoxylates, secondary alcohol ethoxylates, octyl phenylethoxylates, polyethylene lauryl ethers and/or sorbitan esters. Further examples of nonionic surfactants are known to the person skilled in the art and can be gathered from the appropriate specialist literature. Examples of surfactants from said groups are as follows:
Pluronic 10300 (from BASF) from the group of propylene oxide/ethylene oxide triblock polymers
Glucopon 650 (from Cognis) from the group of alkyl polyglycosides
Tergitol NP 7 and Tergitol NP 9 (from DOW Chemicals) from the group of nonyl phenylethoxylates
Tergitol 15 S7 and Tergitol 15 S9 (from DOW Chemicals) from the group of secondary alcohol ethyoxylates
Triton X45 and Triton X114 (from DOW Chemicals) from the group of octyl phenylethoxylates
Brij 30 from the group of polyethylene lauryl ethers
Tween 20 from the group of sorbitan esters

In addition to diverse novel structural elements for the design of diagnostic, microfluidic sample holders, the invention present here also describes the general three dimensional design of such sample holders with regard to the degree of hydrophilization of various functional levels. Finally, gradients of the free surface energy are proposed for optimizing the fluidics and the stop functions. It may at first sound contradictory that the distributor channels and/or inlet channels are also partially, but mostly predominantly—with exception of the capillary bottom—of hydrophobic design, since they cannot be wetted by aqueous media without additives. However, this is deliberate. If a low concentration of a suitable surfactant is added to the sample medium as described above, the fluid has sufficiently free surface energy to wet hydrophobic structures. Nonionic surfactants described chiefly come into consideration for diagnostic purposes, since they are at most slightly toxic. As already mentioned, nonionic surfactants are used as additives in many diagnostic and biotechnological methods, but they are chiefly widespread as emulsifiers or solubilizers in pharmaceutical products, or also additionally as wetting agents in detergents, cleaning agents, coloring media etc. These substances, which are chemically very heterogeneous, are mostly of asymmetric design, that is to say they have, for example, a hydrophilic head and a hydrophobic tail. However, there are also symmetrically designed copolymers (EO/PO compounds) with a hydrophobic core and hydrophilic ends. However, not all surfactants have good wetting properties. These are virtually all substances that are used as emulsifiers (low HLB number=hydrophilic/lipophilic balance) or solubilizers (high HLB number). Good wetting agents are substances with an HLB number between 9 and 13, such as the surfactants described above. Again, there are unsuitable ones among the substances, since they are high foaming compounds. Compounds are suitable that have an optimum wetting effect in conjunction with as low a concentration as possible, and do not foam, or do so only slightly (see the abovedescribed substances). One property of good wetting agents is that they come out of a solution at the interface between fluid and solid surface, and are absorbed at the surface. Thus, the concentration in the fluid decreases in proportion to the wetted surface until a critical limit is undershot. The described suitable substance Pluronic 10300 from BASF is capable at a 0.03% concentration of Pluronic 10300 in aqueous media of providing the fluid with sufficient free surface energy to wet the distributor channels. In this case, the fluid firstly flows much more slowly through the channels than in a structure in which the channels are of completely hydrophilic design. At the inlet edge (in the capillary) into a test depression (for example reaction chamber), the fluid then strikes an interface of hydrophobic structures above and hydrophilic structures below. The capillarity downward is now preferred not only because of the vertical capillary, but also owing to the energy conditions. The fluid quickly reaches the bottom, wets the latter and rises rapidly in the test depression (for example reaction chamber) until it reaches the hydrophobic layer (the first in the vicinity of the inlet edge/inlet capillary). The wetting of the remaining surface is slowed down in this case. The wetting of the sealing layer takes place from the inlet edge/inlet capillary in the direction of the vent capillary with so much retardation that all air can escape. The liquid, which now contains only a low concentration of surfactant, is stopped in the completely hydrophobic vent structure by the combination of the structural elements and the conditions, which are unfavorable in terms of energy. It was even possible given another substance (Tergitol NP9) to fill an untreated, that is to say hydrophobic, sample holder made from polystyrene in a fault free fashion when the sealing layer has the properties described at the beginning, that is to say is still more hydrophobic than the sample holder. When the sealing layer was more hydrophilic (for example acrylate adhesives), the fluid capillarized along the edges between sample holder and sealing layer. The test depressions (for example reaction chambers) were not filled.

Finally, the invention provides a kit for microbiological diagnostics, immunology, PCR (polymerase chain reaction), clinical chemistry, microanalytics and/or the testing of active substances including a sample holder according to the invention.

Further details and features of the invention emerge from the following description of preferred exemplary embodiments in conjunction with the subclaims. Here, the respective features can be implemented on their own or separately in combination with one another. The invention is not limited to the exemplary embodiments.

The exemplary embodiments are illustrated schematically in the figures. Identical reference numerals in the individual figures designate in this case identical elements or elements of identical function or corresponding to one another with regard to their function:

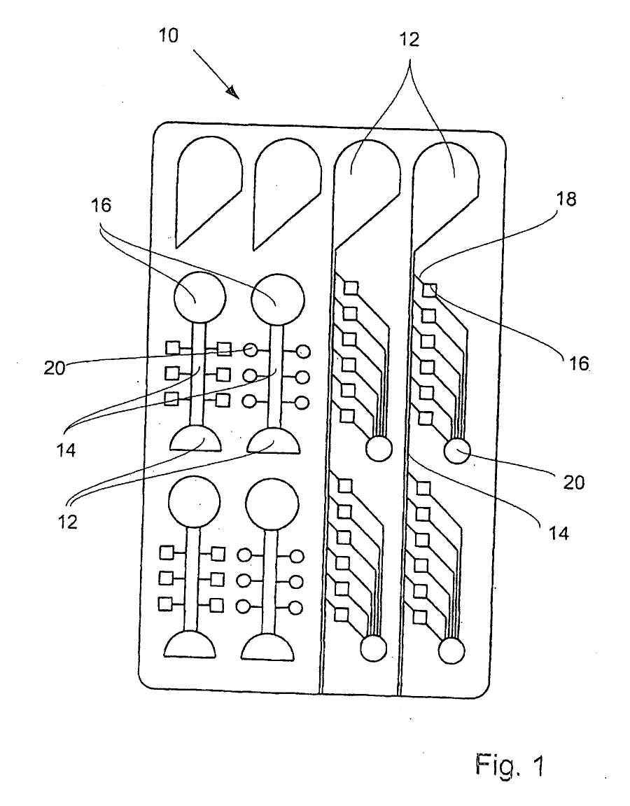

FIG. 1 shows a plan view of a schematic of a sample holder.

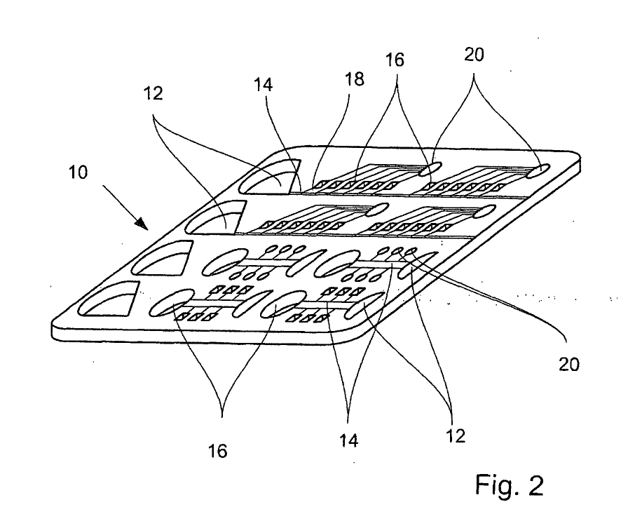

FIG. 2 shows a perspective side view of a schematic of a sample holder.

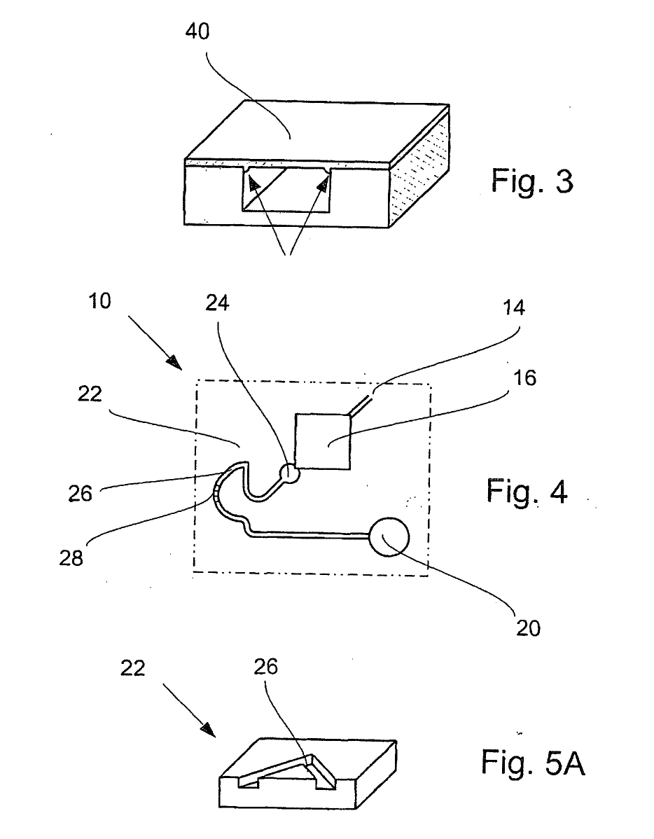

FIG. 3 shows a schematic of the production of microbeads at the transition between sidewalls and cover element.

FIG. 4 shows a schematic of an advantageous embodiment of the sample holder.

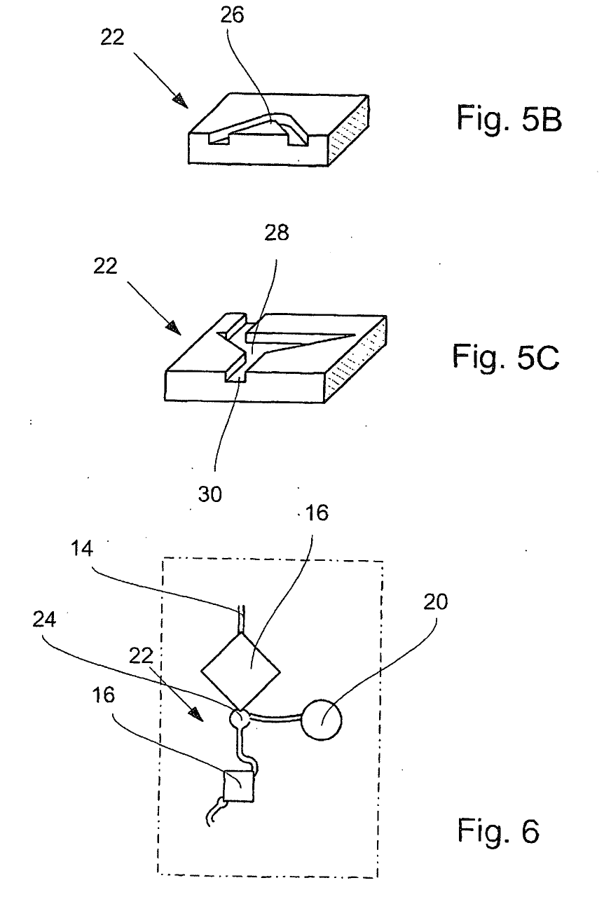

FIGS. 5A-5C show advantageous refinements of the additional structure.

FIG. 6 shows a schematic of an advantageous embodiment of the sample holder for carrying out consecutive assays.

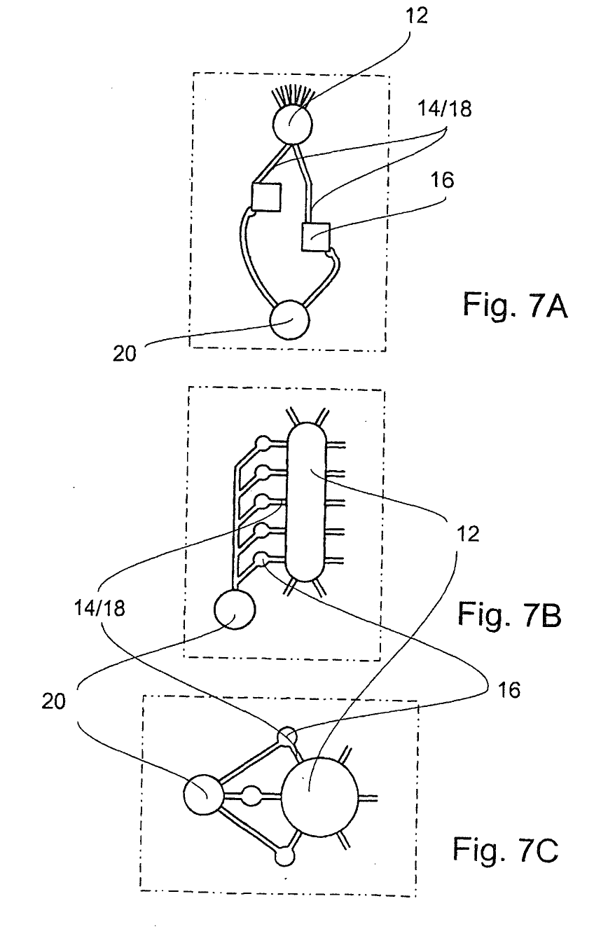

FIGS. 7A-7C show advantageous arrangements of the sample holder.

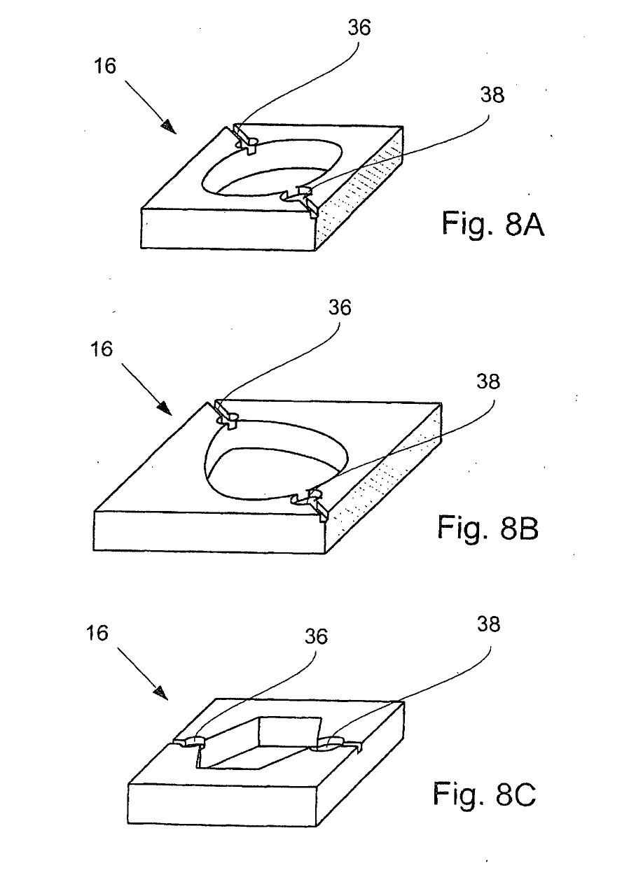

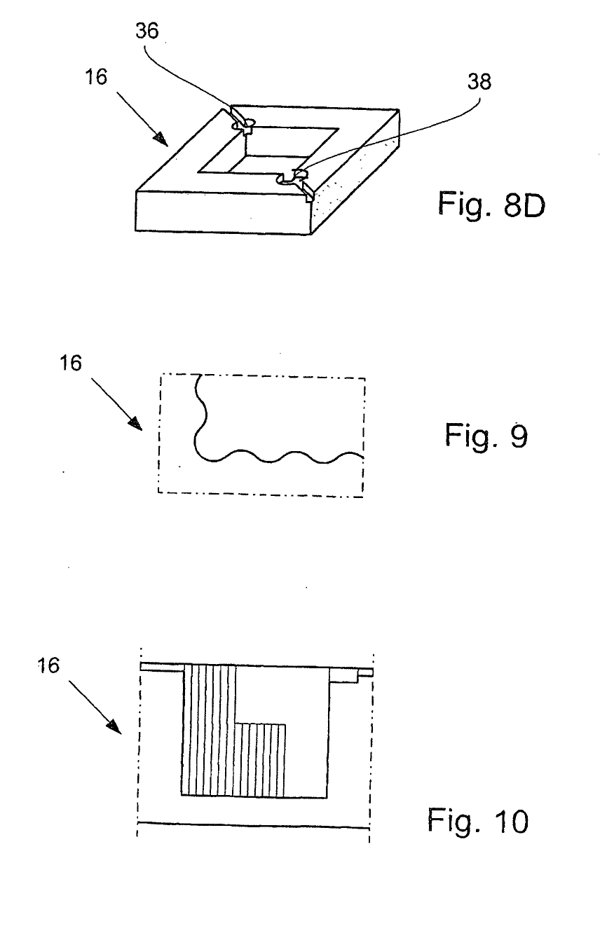

FIGS. 8A-8D show advantageous refinements of the reaction chamber.

FIG. 9 shows a schematic of the sidewalls of the reaction chamber.

FIG. 10 shows a schematic of the extent of the sidewalls of the reaction chamber.

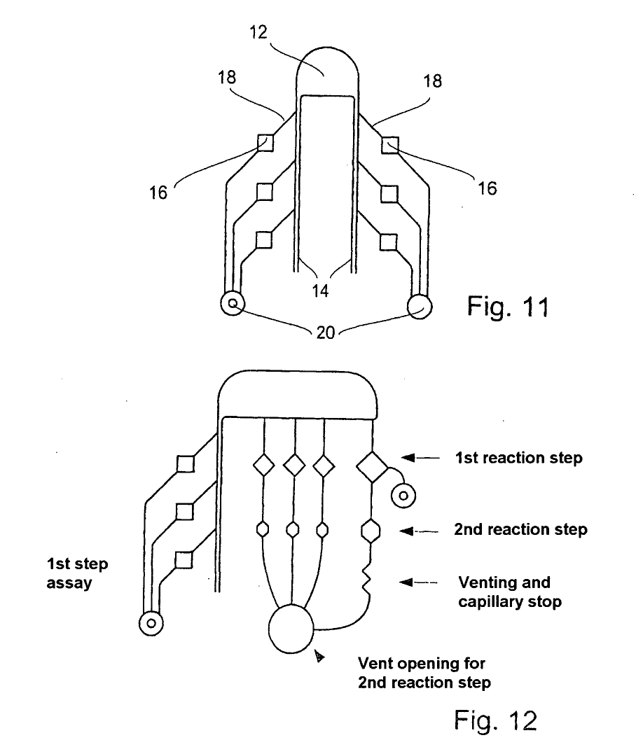

FIG. 11 shows a schematic of an advantageous arrangement of the sample holder for (multiparameteric) one-step assays.

FIG. 12 shows a schematic of an advantageous arrangement of the sample holder for two-step assays.

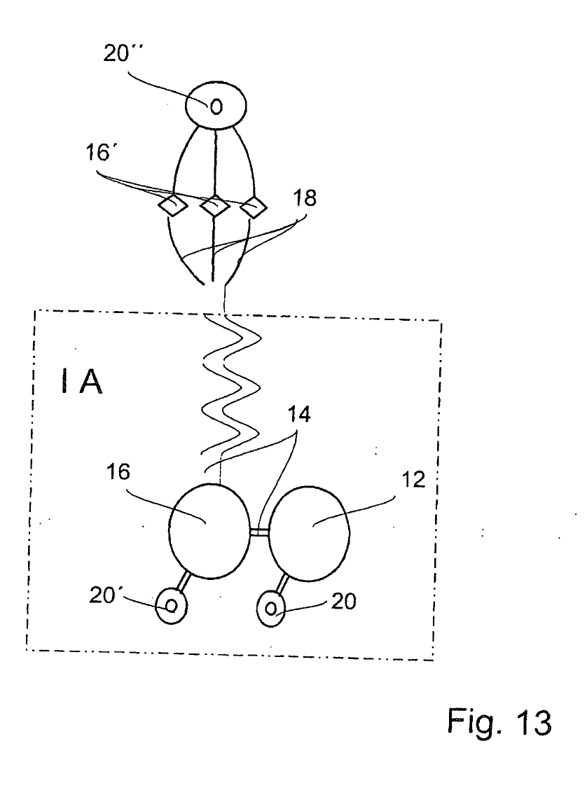

FIG. 13 shows a schematic of an advantageous arrangement of the sample holder for an PCR.

Numerous multiplications and developments of the exemplary embodiments described can be implemented within the scope of the invention.

Range specifications always cover all—not named—intermediate values and all conceivable subintervals.

FIG. 1 shows a plan view of a schematic of a sample holder (10). Various refinements of the structures are to be seen independently of the reaction that is to be carried out in particular (left and right halves of the sample holder). Likewise, the individual structures are formed in different geometric ways depending on requirement in each case. To be seen in concrete terms, are the sample receiving chambers (12), from which a distributor channel (14) extends, the distributor channel (14) in the left-hand half of the sample holder (10) directly connecting the sample receiving chamber (12) to the reaction chamber (16), while in the right-hand half of the sample holder (10) an inlet channel (18) which branches off from the distributor channel (14) is also interposed between the sample receiving chamber (12) and reaction chamber (16). Branching off from these reaction chambers (16) are vent structures or vent capillaries that respectively open into the vent openings (20). The sample holder (10) illustrated in FIG. 1 constitutes the basic structure of a microfluidic sample holder without exhibiting the inventive further additional structures, which are at least partially of hydrophobic design. These additional structures are described in the following figures.

FIG. 2 shows a perspective side view of a schematic of the sample holder (10) according to FIG. 1. To be seen, anew, are the variously designed sample receiving chambers (12), the distributor channels (14) branching off therefrom, as well as the reaction chambers (16) and the vent openings (20). Inlet channels (18) branch off from the distributor channel (14) in the right-hand half of the sample holder (10).

FIG. 3 shows a schematic of the production of microbeads at the transition between sidewalls and cover element. It has emerged that advantageous effects occur during the sealing of the sample holder with a cover element (40) when the cover element is provided on one side with an adhesive layer. When this adhesive layer is a cohesion adhesive, undesired capillary forces can be suppressed between sidewalls and covering. At the site, that is to say between sidewalls and covering, “microbeads” are formed (marked by arrows in FIG. 3), and they ensure that capillarization between sidewalls and cover element is suppressed.

FIG. 4 shows a schematic of an advantageous embodiment of the sample holder. To be seen in this figure are the additional structure (22), which is arranged in this case between the reaction chamber (16) and the vent opening (20). In FIG. 4, the additional structure (22) is a semicircular depression (24) that is located diagonally opposite the distributor channel (14). Preceding from this semicircular depression (24) is a further capillary (20), which is designed as a sharp edged element (28) in FIG. 4.

FIGS. 5A-5C show advantageous refinements of the additional structures (22). Here, FIG. 5A illustrates a structure of zigzag design, while FIG. 5B illustrates a structure angled away sharply and exhibiting an angle of ≧90 degrees. Illustrated in FIG. 5C is a sharp edged element (28) having a changing structural depth and from which there proceeds a further capillary (30) which can open directly or via a neighboring structure into a terminal depression having a valve function.

FIG. 6 shows a schematic of an advantageous embodiment of the sample holder for carrying out consecutive assays. To be seen are the distributor channel (14), the two reaction chambers (16), of different size, the additional structure (22), which here is designed as a semicircular depression (24), as well as the vent capillary that connects the semicircular depression (24) to the vent opening (20). In the case of such an arrangement, the first reaction chamber (16), the larger reaction chamber in FIG. 6, is filled as soon as a sample is placed thereon so that the first step of a reaction can run. The reason for this is that the open vent opening (20), which lies to the side of the first reaction chamber (16), permits only the larger reaction chamber (16) to be filled. Only once the vent system in the second reaction chamber (16) (which is not illustrated in FIG. 6) is opened, can the sample flow out from the larger reaction chamber (16) into the second reaction chamber (16), which has a lesser volume. The second step of a reaction can then follow therein.

FIGS. 7A-7C show advantageous arrangements of the sample holder. To be seen in FIG. 7A is a “jellyfish-like” arrangement of the sample holder, the head of the jellyfish being intended to represent the sample receiving chamber (12), while the “tentacles” take over the function of the distributor and/or inlet channels (14/18). Furthermore, FIG. 7A illustrates the reaction chamber (16) and the vent opening (20).

FIGS. 7B and 7C illustrate further possible refinements of the sample holder according to the invention, wherein the sample receiving chamber (12) is once again formed centrally in the shape of a circle (in FIG. 7C) or an elongated structure (FIG. 7B), and the distributor and/or inlet channels (14/18) depart therefrom. The reaction chamber (16) and the vent opening (20) are arranged around the sample receiving chamber (12).

FIGS. 8A-8D show advantageous refinements of the reaction chamber. Here, the cross sections of the reaction chambers (16) exhibit a round (FIG. 8A), a pear-shaped (FIG. 8B), a hexahedral (FIG. 8C) or a rectangular (FIG. 8D) shape. To be seen, furthermore, are an inlet capillary (36) and an indentation (38) arranged diagonally opposite the inlet capillary (36).

FIG. 9 is a schematic illustration of the sidewalls of the reaction chamber. The sidewalls of corrugated design, which act as vertical capillaries in conjunction with an enlargement of the surface by the corrugated structure, are to be seen. Owing to this arrangement, when sample substances are introduced into solution they can be distributed quickly and uniformly over a relatively large surface, and thus accelerate the drying process while simultaneously “relieving” the inlet capillaries.

FIG. 10 shows the schematic of the extent of the sidewalls of the reaction chambers. The corrugated structure of the sidewalls can extend over various regions. It is illustrated in FIG. 10 that the corrugated structure extends in the vicinity of the inlet capillaries from the bottom up to the cover, while it is entirely lacking in the vicinity of the vent structure. It has proved that in the case of such a distribution of the corrugated structure the incoming fluid in the region of the inlet capillary and of the continuous corrugated structure wets the cover element, and the recondition effect in the remaining part is so strong that the air has enough time to escape.

### 1. One-Step Assay

A simple design composed of a sample receiving chamber, distributor and/or inlet channels, reaction chambers and vent openings (including the feeding structures) suffices for (multiparametric) one-step assays (antigen detection, microbiological tests etc.). The terminal “vent valves” or vent openings are opened in this case (see FIG. 11).

### Methods for One-Step Assays—Antibody Test

If the vent opening is firstly left closed in the case of a sample holder as in FIG. 11, it is possible to carry out a first reaction step in the sample receiving site (12). This is to be illustrated by way of example by a simple antibody test for detecting pathogens of respiratory track diseases for example. In the reaction chambers (16) of one (left-hand) side there are located magnetic particles coated with antihuman IgA or antihuman IgM, as well as fluorescence marked antigen (for example RSV, influenza etc.), while on the right-hand side only the marked antigens are located. Located in the sample receiving chamber (12) are paramagnetic nanoparticles, coated with antihuman IgG, in a concentration sufficient to bind all the IgG from a 1:10 to 1:50 diluted serum sample. Once this first incubation step is concluded, a strong magnetic field is applied to the sample receiving chamber (12), and the vent opening (20) on the left-hand side is opened. The sample now flows into the reaction chambers (16), and IgA and IgM, respectively, bind to the magnetic particles. If specific IgM or IgA are present, these bind with the appropriately marked antigens. The reaction can be evaluated by 3D fluorescence scanning or other optical detection systems. Once the reaction chambers (16) on the left-hand side are filled, the magnetic field is switched off, and the right-hand vent opening (20) is opened. The sample with the nanoparticles now flows into the reaction chambers (16) of the right-hand side, and after a further incubation step it is now possible to detect specific IgG antibodies in a comparable way. The method is also suitable for IgG subclasses or, appropriately modified, for IgE determinations, that is to say for allergy determinations, for example.

### 2. Two-Step Assays

(With or without One-Step Assay)

FIG. 12 shows a design for carrying out two-step assays. The design can also provide structures for simultaneously carrying out one-step assays. Sample preparation can also take place, if appropriate, when all valve functions (that is to say all vent openings) are closed. Two-step assays are known, for example, from clinical chemistry when, for example, the first reaction step presupposes an enzymatic reaction whose end product is detected with the aid of a reagent that is incompatible with the enzyme reaction. Another example would be reactions from coagulation diagnostics, in the case of which only the surplus of an analyte in the sample is to be detected, that is to say there is a need to inactivate a certain defined fraction of an analyte in a first step, no matter of what type. In these assays, the chamber for the first step is approximately five times as large as the test depression for the second step, which is not initiated until the terminal vent opening is opened. Owing to the different size, it is ensured that only material for which the first step has been performed reaches the second chamber.

### Special Case: Antibody Detection

When the sample holder is to be used to detect antibodies and beads which are coated with antigen, for example, are located in the first chamber, the first chamber is much smaller than the second chamber. The latter then is used only as “litter bin” for the samples and washings. These steps can easily be controlled via the terminal vent opening by opening and closing.

### Special Case: PCR Sample Holder

FIG. 13 shows the special case of a PCR sample holder. The sample holder permits the carrying out of a PCR, if appropriate also the isolation of DNA/RNA and the subsequent detection of the targets, if appropriate after a second specific PCR. The sample holder is approximately 2-3 mm thick and of hydrophobic design up to an intermediate layer 50 μm-100 μm thick. The bottom of the sample holder consists in the front part (I A) of a thin plastic coated metal foil, and in the rear part of thermostable plastic. The cover is an adhesive coated highly elastic film. The sample receiving chamber (12) (20-100 μl) is used for sample preparation. It is ventilated during injection of the sample via a simple vent channel and an opened vent opening (20). The depression (12) can contain all reagents that are required for the isolation. Materials that should not be transferred to a subsequent process are bound to a solid phase (for example magnetic particles). Once the isolation is concluded, the vent opening (20′) is opened and the vent opening (20) is mechanically closed, while the sample (reinforced by heating, if appropriate) flows into the reaction chamber (16) via a distributor channel (14). All the reagents for carrying out a PCR are located in the reaction chamber (16), partially bound on solid phases if necessary for optimizing the method. After the PCR (multiplex or specific) has been performed, the vent opening (20″) is opened and the amplificate can pass into the reaction or detection chambers (16′) via channel systems. The distributor channel (14) is firstly of meandering shape and completely hydrophobic, subsequently tapers, although becoming deeper, and comes to lie in a hydrophilic layer in its lower part. The still narrower inlet channels (18) are likewise hydrophobic in the lower part. The meandering structure of the hydrophobic distributor channel (14) and the closed valve or the closed vent structure (20″) prevent premature transfer from the reaction chamber (16) into the detection chambers (16′). During the PCR, the vent openings (20) and (20′) are also closed from outside. The vent capillaries are individually connected to the vent opening (20″) and contain capillary stop structures, as already described elsewhere. As an option, a second PCR can be carried out, or the detection can be performed directly in the chambers (16′), which can have various geometric shapes. It is possible to this end, in turn, for traps or detection probes (for example hairpins) to be bound to beads. It is not intended here to go into more detail on the multiplicity of variations.

### REFERENCE SYMBOLS

10 Sample holder
12 Sample receiving chamber
14 Distributor channel
16 Reaction chamber
18 Inlet channel
20 Vent opening
22 Additional structures
24 Semicircular depression
26 Capillary
28 Sharp edged element
30 Additional capillary
36 Inlet capillary
38 Indentation
40 Cover element

1-31. (canceled)

32. A sample holder having
at least one sample receiving chamber for a sample fluid,
at least one distributor channel that is connected to the at least one sample receiving chamber, at least one distributor channel extending from each sample receiving chamber,
at least one reaction chamber into which, if appropriate, an inlet channel branching off from the at least one distributor channel opens, and
at least one vent opening for each reaction chamber,
wherein between the sample receiving chamber, distributor channel, reaction chamber, inlet channel, present if appropriate, and/or vent opening the sample holder has at least one further additional structure that is at least partially of hydrophobic design, and
in its upper region the distributor channel and/or the inlet channel lies in a plane with the vent opening, and is of substantially hydrophobic design in this region, whereas in its lower region, lying beneath the vent opening, it is at least substantially of hydrophilic design, and
in its upper region lying in a plane with the hydrophobic part of the distributor channel and/or inlet channel the reaction chamber is of substantially hydrophobic design, whereas in its lower region, lying beneath the hydrophobic region of the distributor channel and/or inlet channel, it is of substantially hydrophilic design or
the reaction chamber is of generally hydrophobic design.

33. The sample holder as claimed in claim 32, wherein in the case of a reaction chamber of substantially hydrophilic design, the hydrophilization increases in layerwise fashion in the lower region of the reaction chamber.

34. The sample holder as claimed claim 32, wherein each further structure has a cross section of approximately 10 μm to approximately 300 μm, preferably approximately 50 μm to approximately 200 μm, in particular approximately 100 μm to approximately 150 μm.

35. The sample holder as claimed in claim 32, wherein the additional structure is a substantially semicircular depression that is preferably arranged diagonally opposite the distributor channel.

36. The sample holder as claimed in claim 35, wherein at least one further capillary extends from the semicircular depression, the further capillary being designed in a fashion sharply angled away, preferably at an angle ≧90°, and/or in a zigzag fashion.

37. The sample holder as claimed in claim 36, wherein extending away from the further capillary is at least one further element, which is substantially sharp edged and has a changing structural depth.

38. The sample holder as claimed in claim 37, wherein at least one further capillary extends away from the element which is substantially sharp edged and has a changing structural depth, the further capillary opening directly or via a neighboring structure into a terminal depression having a valve function.

39. The sample holder as claimed in claim 32, wherein the distributor channel, which is connected to the sample receiving chamber, is of the meandering design.

40. The sample holder as claimed in claim 32, wherein a number of vent openings, distributor channels, if appropriate inlet channels, reaction chambers and/or additional structures are arranged around the sample receiving chamber or parallel thereto.

41. The sample holder as claimed in claim 32, wherein the reaction chamber has a vertical extent of approximately 500 μm to approximately 3 mm, preferably approximately 1 mm to approximately 2.5 mm, in particular approximately 1.5 mm to approximately 2 mm.

42. The sample holder as claimed in claim 41, wherein the edge length of the reaction chamber has an average of approximately 300 μm to approximately 1 mm, preferably approximately 500 μm to approximately 750 μm, in particular 500 μm to approximately 600 μm.

43. The sample holder as claimed in claim 41, wherein the cross section of the reaction chamber is of essentially round, pear shaped, hexahedral, octahedral and/or rectangular design.

44. The sample holder as claimed in claim 41, wherein the reaction chamber has a vertically running and substantially rounded inlet capillary in the bottom region.

45. The sample holder as claimed in claim 44, wherein the inlet capillary has a radius of approximately 5 μm to approximately 50 μm, in particular approximately 10 μm to approximately 20 μm.

46. The sample holder as claimed in claim 41, wherein the reaction chamber (16) has an indentation that is preferably arranged diagonally opposite the inlet capillary and leads to at least one vent opening.

47. The sample holder as claimed in claim 43, wherein the reaction chamber has at least one rounded corner.

48. The sample holder as claimed in claim 41, wherein the reaction chamber (16) has sidewalls of substantially smooth and/or corrugated design.

49. The sample holder as claimed in claim 32, wherein the sample holder is covered in a fluid-tight fashion by a cover element.

50. The sample holder as claimed in claim 49, wherein the cover element is a film that is provided on one side with an adhesive layer of suitable thickness.

51. The sample holder as claimed in claim 50, wherein the film and/or adhesive is a heat activatable and/or pressure sensitive film or adhesive.

52. The sample holder as claimed in claim 50, wherein the film is applied under pressure, preferably at approximately 2 to 5 bars, by means of rolls such that the sample holder has a substantially gapless covering.

53. The sample holder as claimed in claim 50, wherein the film is a fluoro-polymer film.

54. The sample holder as claimed in claim 51, wherein the adhesives are cohesive adhesives.

55. The sample holder as claimed in claim 54, wherein hydrophobic adhesives are concerned.

56. The sample holder as claimed in claim 55, wherein the adhesive is a silicone, rubber, silicone rubber, and/or fluoropolymer adhesive.

57. A method for analyzing at least one sample substance, wherein a sample medium has at least one surfactant added to it and is applied to a sample holder as claimed in claim 32.

58. The method as claimed in claim 57, wherein the surfactant is a nonionic surfactant.

59. The method as claimed in claim 58, wherein the nonionic surfactant is a substance with an HLB number between approximately 9 and approximately 13.

60. The method as claimed in claim 59, wherein the tenside is a propylene oxide/ethylene oxide triblock polymer, an alkyl polyglycoside, a nonylphenylethoxylate, a secondary alcohol ethoxylate, an octyl phenylethoxylate, a polyethylene lauryl ether and/or a sorbitan ester.

61. A kit for microbiological diagnostics, immunology, PCR (polymerase chain reaction), clinical chemistry, microanalytics and/or the testing of active substances including a sample holder as claimed in claim 32.
